(19) United States
(12) Patent Application Publication
(10) Pub. No.: US 2022/0310210 A1
(43) Pub. Date: Sep. 29, 2022

(54) METHOD OF TESTING AND FITTING THE DIHEDRAL ANGLE PARAMETERS IN FORCE FIELD

(71) Applicant: SHENZHEN JINGTAI TECHNOLOGY CO., LTD., Guangdong (CN)

(72) Inventors: Dong FANG, Guangdong (CN); Guo WANG, Guangdong (CN); Mingjun YANG, Guangdong (CN); Jian MA, Guangdong (CN); Shuhao WEN, Guangdong (CN); Lipeng LAI, Guangdong (CN)

(73) Assignee: SHENZHEN JINGTAI TECHNOLOGY CO., LTD., Guangdong (CN)

(21) Appl. No.: 17/293,923

(22) PCT Filed: Jun. 15, 2020
(86) PCT No.: PCT/CN2020/096122

§ 371 Date: May 14, 2021

(51) Int. Cl. G16C 10/00; G06N 10/20

(52) U.S. Cl. CPC G16C 10/00; G06N 10/20

(57) ABSTRACT

The present invention provides a method for testing and fitting the dihedral angle parameters in force field. The method first generates some representative conformations, and then compares the results of force field and quantum mechanics methods using these structures. If the results meet the predefined standards, the process ends; otherwise the molecule will be cut into small-size molecular fragments with only one flexible dihedral angle in each fragment. The dihedral angles will be scanned. And results of force field and quantum mechanics will be compared for each scanned flexible dihedral angle to find out those that do not meet the standards, and their parameters will be selected for further fitting. After new dihedral angle parameters are obtained, apply them to the original series of conformers of the whole molecule for validation. If results meet the standards, complete the whole process of testing and fitting poorly performing dihedral angle parameters.

### TECHNICAL FIELD

The invention pertains to the field of molecular mechanics, and specifically relates to a method for testing and fitting the dihedral angle parameters in force field, which is suitable for evaluating the dihedral angle parameters in molecular force field, and correcting poorly performing parameters by fitting.

### BACKGROUND TECHNOLOGY

Molecular mechanics is widely used in many fields such as drug design due to its advantage in speed and reliable accuracy. Molecular mechanics is based on formulas that describe molecular properties (such as energy) and corresponding parameters, and the parameters are called molecular force fields. The commonly used force field energy is defined as: E=E_{bond}+E_{angle}+E_{dihedral}+Eᵢₘₚᵣₒₚₑᵣ+Eₑₗₑ+E_{vdw}, where E_{bond} is the energy determined by the bond length of two connected atoms, and E_{angle} is the energy of the angle determined by three connected atoms, E_{dihedral} is the energy of the dihedral angle determined by four connected atoms, Eᵢₘₚᵣₒₚₑᵣ is the out-of-plane bending energy of four atoms that are expected to be in the same plane, Eₑₗₑ is the electrostatic energy between two atoms, and E_{vdw} is the van der Waals energy of two atoms. Corresponding to the energy terms above, bond, angle, dihedral angle, out-of-plane bending, charge and van der Waals parameters constitutes force field. Regarding of quantity and flexibility, the dihedral angle parameter is much more diverse than other parameters. Therefore, the quality of the dihedral angle parameter is very important to the overall quality of the force field.

The development of the molecular force field is generally based on smaller molecular fragments, Quantum mechanics calculations are performed and their corresponding results (usually energy) are used as the target for fitting to obtain a series of force field parameters. Evaluation of the force field parameters usually requires quantum mechanics calculation of other small molecules that are not in the training set as the standard. A large amount of quantum mechanics calculation on small-size molecules is feasible. The quantum mechanics method is usually density functional theory or high-precision methods based on perturbation theory. These small molecules generally have 1-2 flexible dihedral angles. The traditional dihedral scanning is to rotate dihedral angles in the range of −180 degrees −180 degrees with certain interval (generally 15 degrees). During the scanning process, the specific dihedral angle is fixed at a specific angle for structural optimization. Finally, the force field is evaluated by comparison with the energy obtained by quantum mechanics.

As mentioned above, the development of force fields is generally based on small molecules, but in practical applications such as drug molecule design, the molecules are often relatively large (with more than 3 flexibility angles), which requires force field parameters, dihedral angle parameters in particular can transfer from small molecules to large molecules. The traditional method for testing and fitting dihedral angle parameters is aforementioned method of dihedral angle scanning on the whole molecule (see FIG. 1). Since large molecules often contain many more flexible dihedral angles, and there may be coupling between dihedral angles, so a combined scan of the coupled dihedral angles is usually required. As a result, for large molecules, traditional methods require a large amount of high-precision quantum mechanics calculation.

### DESCRIPTION OF THE INVENTION

In view of the above technical problems, the present invention provides a testing and fitting method suitable for the dihedral angle parameters of large-size molecules. This method requires less calculation than the traditional method.

The specific technical solutions are:

A method of testing and fitting the dihedral angle parameters in force field

First, generate some representative conformations. These structures represent a variety of values of the flexible dihedral angles in the molecule. Compare results of force field with quantum mechanics methods. If they meet the standards, it is deemed that the force field parameters are satisfactory and the process ends.

If they do not meet the standards, further cut the whole molecule into molecular fragments with only one flexible dihedral angle, scan the dihedral angle and compare force field's results with quantum mechanics for each flexible dihedral angle to find out those on which parameters do not perform well and fit their values. After new dihedral angle parameters are obtained, use the original series of conformations of the whole molecule for validation. If they meet the standard, end and complete the whole process of testing and fitting of poorly performing dihedral angle parameters. If they do not meet the standards, perform the dihedral scan of the whole molecule for the poorly performing flexible dihedral angles.

The specific steps are:

(1) For a large molecule, first use rdkit to generate 500 conformations for each molecule (the specific command is rdkit. EmbedMultipleConfs(mol, 500)). Use rdkit's own UFF force field for structural optimization (rdkit. UFFGetMoleculeForceField(mol).Minimize( )) and calculate the angle of each flexible dihedral angle of each structure. According to the angle distribution of the flexible dihedral angles, 30 structures are selected to cover a variety of areas from −180 degrees to 180 degrees. In practice, the structure with lower energy occupies a larger proportion. Therefore, for structures with the same flexible dihedral angle, priority is given to those with lower energy;

(2) Use high-precision quantum mechanics methods (such as B3LYP/6-31G(d)) in software (such as PSI4) to further optimize the structure in step (1), and get the corresponding energy as E_{QM}. In addition, these structures are optimized with the force field that needs to be tested, and the corresponding energy is obtained as E_{MM};

(3) The two sets of energies obtained in step (2) are linearly fitted for each molecule (python scipy module,_, _, R,_, ΔE=scipy.stats.linregress(E_{QM}, E_{MM}), to get The Pearson correlation coefficient R and energy deviation dE of the two sets data. If R is greater than the first threshold and dE is less than the second threshold, it is preferable in this invention that the R is greater than 0.7 and dE<2.0 kcal/mol (The two thresholds can be lowered or raised according to the specific requirements of the user. The standards in the following steps are the same.), which indicates the good performance of parameters on this molecule, the process ends. Otherwise, go to step (4);

(4) Cut the molecules that have entered this step from step (3) into smaller fragments, and each fragment contains a flexible dihedral angle (applicable to rdkit's rdkit. Chem.rdmolops.FragmentOnBonds( )function). Perform the traditional dihedral scan of these fragments and compare results of force field with quantum mechanics. For dihedral angles on which parameters do not perform well (the correlation coefficient R between the two is less than 0.7 or the energy deviation dE is greater than 2.0 kcal/mol), use the quantum mechanics data to fit and obtain new dihedral angle parameters.

(5) Using the newly fitted dihedral angle parameter obtained in step (4), repeat the force field calculation in step (2) to obtain new energy E_{MM′}. The quantum chemistry data E_{QM} previously calculated in step (2) is linearly fitted with E_{MM′}, to obtain the correlation coefficient R′ and energy deviation dE′. If R′ is greater than 0.7 and dE′ is lower than 2.0 kcal/mol, it indicates the good performance of new parameters, and the process ends. Otherwise, go to step (6);

(6) Perform a traditional dihedral scan of the whole molecule for the parameters that still perform poorly in steps (4)-(5). The dihedral angles on which parameters do not perform well in step (4) are scanned and further refitted. Apply newly fitted parameters to initially generated structures and compare results of force field with quantum mechanics results E_{QM} of step (2). If R is greater than 0.7 and dE is less than 2.0 kcal/mol, the process ends. Otherwise, continue to scan other flexible dihedral angles and fit the relevant dihedral angle parameters.

The method for testing and fitting the dihedral angle parameters of the force field provided by the present invention has the following technical advantages:

The new method for testing and fitting dihedral angle parameters for large-size molecules proposed by the present invention saves more calculations than the traditional dihedral scanning of the whole molecule. By combining with the traditional process, the testing and fitting of the dihedral angle parameters of the force field with large-size molecules can be completed with less computational resource consumption.

### DESCRIPTION OF THE DRAWINGS

FIG. 1 shows the current traditional large-size molecule testing and fitting process.

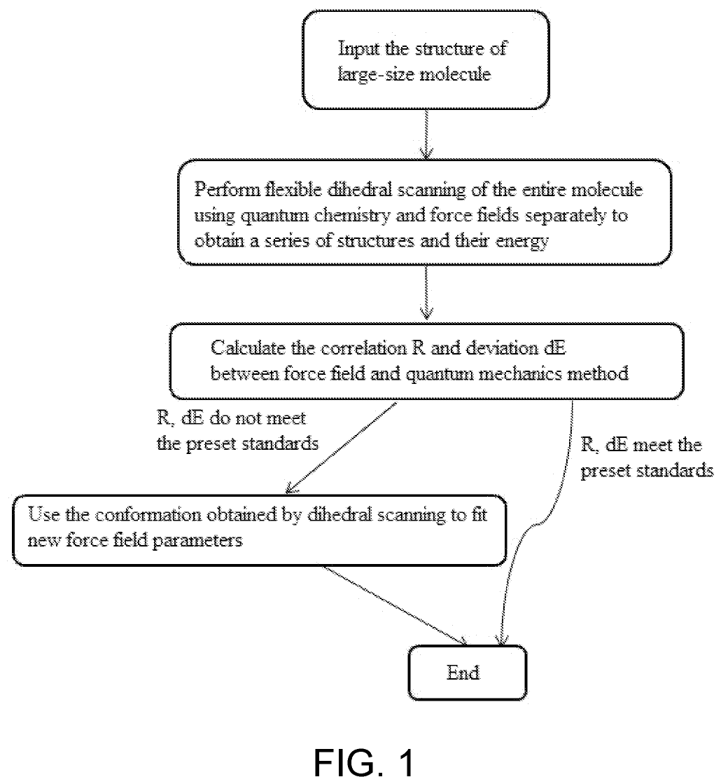

FIG. 2 is a flow chart of the present invention. The lower left part is a general method based on dihedral scanning of the whole molecule. After being combined with the new method of the present invention, these steps only need to be applied to the dihedral angles that do not meet the preset standard in previous steps.

### DESCRIPTION OF THE PREFERRED EMBODIMENTS

The specific technical solutions of the present invention are explained in conjunction with the embodiments.

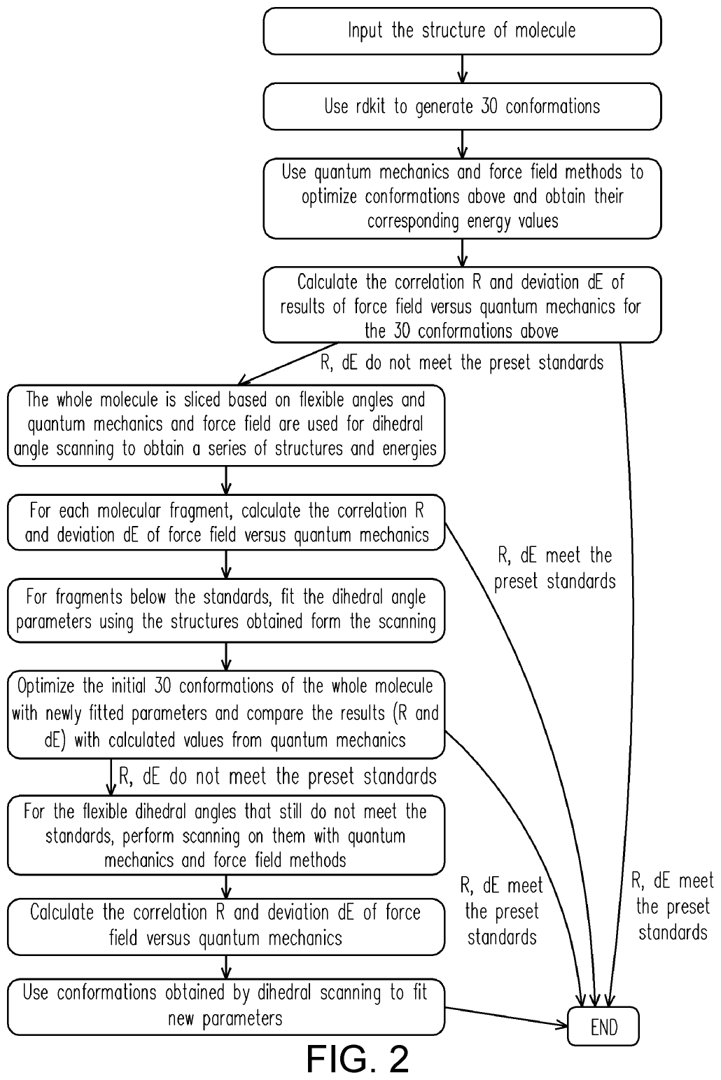

The process shown in FIG. 2:

Take the large-size molecule (SMILES:COC1=C(OCCCC2=NC3=CC=CC=C3 [N]2C)C=C4N=CN=CC4=C1) as an example, the molecule has 6 flexible dihedral angles, the traditional dihedral scanning of the entire molecule requires optimization of 600 conformations. Each structural optimization (quantum chemical method using B3LYP/6-31G(d)) requires 2 hours CPU time (calculation time on a single CPU core); compared with the calculation time of quantum mechanics, the time for force field calculation and force field fitting can be ignored (in the following method, only the time for quantum mechanics calculation will be compared), so a total of 1200 hours of CPU time is required.

According to the method designed in the present invention, first use rdkit to generate 500 conformations, and determine 30 final conformations via structural screening. The amount of calculation required for this process is negligible.

Then the 30 conformations are optimized by quantum mechanics calculations, and the time required is 2 hours*30=60 hours. The force field method (here the GAFF2 force field is used) is used to optimize the structure, and the time can be ignored. So, the CPU time required for this step is 60 hours.

Comparing the energy E_{QM} of quantum mechanics and the energy E_{MM} of the force field, the Pearson correlation coefficient R is 0.76 and the deviation dE is 1.6 kcal/mol. This complies with the standard set by the present invention. Therefore, the performance of the tested force field meets the predetermined standard, and no further fitting is required. The calculation of the whole method is 60 hours CPU time, which is significantly less than the traditional 1200 hours.

In order to compare the calculation amount of the following steps, this embodiment continues to the next step. Cut this molecule into 6 molecular fragments. Perform dihedral angle scan from −180 degrees to 180 degrees and 15 degrees for each of them, so that 24 conformations need to be calculated for each molecular fragment, and a total of 24*6=144 conformations need to be calculated. The average CPU time required for each molecular fragment calculation is 0.16 hours, so the total time required for this step is 144*0.16=23 hours. In this embodiment, the quantum mechanics data of these molecular fragments are used for fitting to obtain six new flexible dihedral angle parameters. Apply the new parameters to the 30 conformations generated in the first step, comparing it with the quantum mechanics data in the first step, and recalculate the energy correlation coefficient and deviation respectively as 0.83 and 1.3 kcal/mol. It can be seen that the fitting based on molecular fragments improves the quality of the dihedral angle parameters. Up to this step, the total calculation time required is 60+23=83 hours, which is far less than the traditional 1200 hours.

In order to demonstrate the entire process, this embodiment selects the relatively poor two dihedral angles among the six dihedral angles to scan the dihedral angle of the whole molecule. Without considering the coupling, the CPU time required is 2 (the number of dihedral angles)*24 (the number of conformations required for each dihedral angle scan)*2 hours=96 hours, and then the two dihedral angle parameters are fitted. The total time required is 96+60+23=179 hours.

According to the comparison of the examples above, the calculation required by the new testing and fitting method of the present invention is far less than that of the traditional dihedral scan of the whole molecule.

1. A method for testing and fitting dihedral angle parameters of force field, comprising the following steps:
generating a series of representative conformations that represent various angles of flexible dihedral angles in a molecule; comparing results of force field and quantum mechanics methods using these conformations; if they meet the preset standards, it is deemed that force field parameters are satisfactory and the process ends; if they do not meet the standards, further cut the whole molecule into molecular fragments that contain only one flexible dihedral angle, scan the dihedral angles, and compare the quantum mechanics' results of each flexible dihedral angle with the force field's results to identify the flexible dihedral angles with large deviation, and fit their parameters; after new dihedral angle parameters are obtained, validate these parameters on initially generated structures of the whole molecule; if they meet the standards, complete the whole process of testing and fitting of poorly performing dihedral angle parameters; if they do not meet the standards, perform dihedral angle scans on the whole molecule for the identified poorly performing flexible dihedral angles.

2. The method for testing and fitting force field dihedral angle parameters according to claim 1, wherein the method comprises the following steps:
(1) for a large-size molecule, first using rdkit to generate 500 conformations for each molecule, optimizing structure of these conformations with an universal force field (UFF) that comes with rdkit, and calculating an angle of each flexible dihedral angle of each structure; selecting 30 structures according to the angular distribution of the flexible dihedral angles, covering various areas from −180 degrees to 180 degrees, preferentially selecting the structures with lower energy;
(2) using quantum mechanics calculation software to further optimize the structure in step (1) and obtaining a corresponding energy as E_{QM}; at the same time, using the force field to be tested to optimize the structures to obtain a corresponding energy E_{MM};
(3) linearly fitting the two sets of energies obtained in step (2) to obtain the Pearson correlation coefficient R and energy deviation dE; if R is greater than a first threshold and dE is less than a second threshold, as it indicates force field parameters' good performance on this molecule, end the process; otherwise, go to step (4);
(4) cutting the molecules that have entered this step from step (3) into smaller fragments, wherein each fragment contains a flexible dihedral angle; performing a traditional dihedral scan of the fragments to compare results of force field and quantum mechanics; for dihedral angles with bad performance, using the quantum mechanics data to fit and obtain new dihedral angle parameters;
wherein bad performance means the correlation coefficient R between the two is less than the first threshold or the energy deviation dE is greater than the second threshold;
(5) using the newly fitted dihedral angle parameter obtained in step (4), repeating the force field calculation in step (2) to obtain a new energy E_{MM}′; using the quantum mechanics data E_{QM} that are previously calculated in step (2) to linearly fit E_{QM} and E_{MM}′ to get the correlation coefficient R′ and energy deviation dE′ with E_{MM}′; if R′ is greater than the first threshold and dE′ is less than the second threshold, which indicates the newly fitted dihedral angle parameters perform well on this molecule, the process is ended; otherwise, go to step (6);
(6) performing the traditional dihedral scan of the whole molecule for the parameters that still perform poorly in steps (4)-(5); scanning the dihedral angles that do not perform well in step (4) first, and then performing dihedral parameter fitting, apply newly fitted parameters to the initially generated structure, and comparing them with the existing quantum mechanics results E_{QM} in step (2); if R is greater than the first threshold and dE is less than the second threshold, the process is ended; otherwise, the scan of other flexible dihedral angles is performed to fit the relevant dihedral angle parameters.

3. The method for testing and fitting dihedral angle parameters according to claim 2, wherein the first threshold is 0.7 and the second threshold is 2.0 kcal/mol.
